SEQUENCE LISTING (2 sequences)

SEQ ID NO: 1 - DNA, 18 bp, unknown
  DNA oligonucleotide (primer DNA)
gtccctgttcgggcgcca

SEQ ID NO: 2 - DNA, 57 bp, Artificial Sequence
  Synthetic DNA oligonucleotide (template DNA)
ctcagacccttttagtcagaatggaaaatctctagcagtggcgcccgaacagggaca

(19) United States
(12) Patent Application Publication
(10) Pub. No.: US 2005/0113331 A1
(43) Pub. Date: May 26, 2005

(54) Compositions and methods for use of antiviral drugs in the treatment of retroviral diseases resistant to nucleoside reverse transcriptase inhibitors

(76) Inventors: Michael Prniak, Pittsburgh, PA (US); John W. Mellors, Pittsburgh, PA (US); Eric Oldfield, Champaign, IL (US); Zev Tovian, Highland Park, IL (US); Julian Mun Weng Chan, Urbana, IL (US)

Correspondence Address: BAKER & BOTTS, 30 ROCKEFELLER PLAZA, NEW YORK, NY, 10112, US

(21) Appl. No.: 10/937,683

(22) Filed: Sep. 9, 2004

Related U.S. Application Data
(60) Provisional application No. 60/501,389, filed on Sep. 9, 2003.

(51) Int. Cl. A61K 31/7072; A61K 31/66

(52) U.S. Cl. 514/49; 514/102

(57) ABSTRACT

The present invention relates to novel compositions comprising an excision-inhibiting bisphosphonate and a nucleoside reverse transcriptase inhibitor. The present invention also relates to methods for preventing or treating retrovirus-related diseases using a composition comprising a bisphosphonate and a nucleoside reverse transcriptase inhibitor. In a specific embodiment, the invention provides methods for preventing or treating AIDS by administering a bisphosphonate-based compound in combination with 3′-azido-3′-deoxythymidine (AZT) to patients infected with AZT-resistant HIV to improve the effectiveness of AZT therapy.

The present application claims the benefit of U.S. Application No. 60/501,389 which was filed on Sep. 9, 2003 and is incoporated by reference herein in its entirety. This invention was made with government support under Contract No. GM 50694 awarded by the National Institutes of Health (NIH). The government may have certain rights in the invention.

### FIELD OF THE INVENTION

The present invention is directed to novel compositions comprising one or more bisphosphonate compounds, and methods for preventing or treating retroviral diseases by administering to subjects having a retrovirus-related disorder, or at risk of becoming infected with a target retrovirus, a pharmaceutical composition comprising such bisphosphonate-containing compositions.

### BACKGROUND OF THE INVENTION

Nucleoside reverse transcriptase inhibitors (“NRTIs”), such as 3′-azido-3′-deoxythymidine (“AZT”), are effective inhibitors of HIV-1 replication. Seven of the fifteen anti-HIV drugs approved by the FDA for clinical use are NRTIs. NRTIs are deoxy nucleotide triphosphate (“dNTP”) analogs that are incorporated during viral replication into growing DNA strands by the viral enzyme, reverse transcriptase (“RT”). Upon attachment of NRTI to the 3′ end of the growing DNA strand, however, further extension of the DNA strand is prohibited because NRTIs lack a 3′ hydroxyl. Viral replication is thereby disrupted. (See, for example, Pamiak et al. (2000) Inhibitors of HIV-1 reverse transcriptase. Adv Pharmacol 49:67-109; Furman et al. (1986) Phosphorylation of 3′-azido-3′-dideoxythymidine and selective interaction of the 5′-triphosphates with human immunodeficiency virus reverse transcriptase. PNAS 83:8333-8337; Goody et al. (1991) Factors contributing to the inhibition of HIV reverse transcriptase by chain-terminating nucleotides in vitro and in vivo. FEBS Lett 291:1-5; Sluis-Cremer et al. (2000) Molecular mechanisms of HIV-1 resistance to nucleoside reverse transcriptase inhibitors (NRTI) Cell Mol Life Sci 57:1408-1422; De Clercq (1994) HIV resistance to reverse transcriptase inhibitors. Biochem Pharmacol 47:155-169).

HIV has exhibited a remarkable ability to develop resistance to virtually all antiviral compounds currently approved for clinical use. A major mechanism for HIV-1 RT resistance to NRTI is “chain terminator excision” which is a phosphorolytic reaction catalyzed by RT that results in the removal of the chain-terminating NRTI from the viral DNA primer 3′-end. HIV develops resistance to NRTIs by removing the terminal NRTI, a process termed “chain terminator excision.” Certain mutations in RT, termed thymidine analog mutations or TAMs, result in higher rates of excision and thus increase clinical resistance to AZT. Most HIV-infected patients that have been previously treated with antiviral drugs are infected with HIV strains that- have AZT-resistant reverse transcriptase. (Hirsch et al. (2003). Antiretroviral drug resistance testing in adult HIV-1 infection: 2003 recommendations of an international AIDS society—USA panel. Clin Infect Dis 37:113-128.) The high incidences of HIV resistance has severely impaired the therapeutic efficacy of NRTI drugs, such as AZT. After prolonged use of AZT therapy, the treatment becomes therapeutically ineffective at reducing viral load.

Therefore, there exists a compelling need to develop new treatment options for retroviral diseases, and particularly finding novel combination therapies for AIDS that combat viral resistance or prolong the effectiveness of currently applied therapies.

### SUMMARY OF THE INVENTION

The present invention provides isolated compositions having bisphosphonate (“BPH”). The isolated compositions encompass variants of bisphosphonate. In particular, the present invention is related to the use of one or more BPHs in combination with one or more nucleoside reverse transcriptase inhibitors (“NRTIs”). Compositions of the present invention may also comprise one or more other antiviral compounds.

The present invention provides compositions having one or more BPHs and a bisphosphonate-recognition site. For example, the composition may comprise a complex of a BPH and a portion of a HIV DNA molecule that binds the BPH. The invention also provides methods for identifying agents that modulate bisphosphonate-mediated NRTI excision by contacting such complexes with test compounds and measuring the effect of the test compounds on bisphosphonate-mediated NRTI excision activity.

The present invention provides methods for preventing or treating AIDS by administering one or more BPHs in combination with AZT to patients infected with AZT-resistant HIV to improve the effectiveness of AZT therapy.

The present invention also provides methods for prolonging the use of or improving the efficacy of drugs that inhibit RT such as AZT. For example, the present invention provides for methods that prolong the use or improve the efficacy of NRTI-based drugs currently used in the treatment of NRTI-resistant antiviral diseases.

The present invention also provides methods for inhibiting retroviral replication in a cell by contacting the cell with an effective amount of one or more BPHs. One or more NRTIs and/or other antiviral compounds may be contacted with the cell prior to, overlapping with, concurrently, and/or after introduction of the BPH.

The present invention also provides methods for inhibiting retroviral replication and/or reducing viral titer in a subject by administering to the subject an effective amount of one or more BPHs. The BPH may be administered prior to, co-administered, concurrently administered with, and/or sequentially administered with, an NRTI and/or other antiviral compound.

The present invention also provides methods for treating a retrovirus-related disorder by administering to a subject in need of such treatment, an effective amount of one or more BPHs, NRTIs, and/or other antiviral compounds.

The present invention also provides methods for inhibiting NRTI excision in a cell infected with a NRTI-resistant retrovirus by contacting the cell with an effective amount of one or more BPHs, NRTIs, and/or other antiviral compounds. For example, such a cell may be infected with a NRTI-resistant HIV.

The present invention also provides assays for identifying a BPH capable of modulating NRTI excision.

The present invention also provides methods for preventing, preventing recurrence of, or reducing the rate of recurrence of, a retrovirus-related disorder in a subject in need of such preventative therapy, by administering to the subject an effective amount of one or more BPHs, NRTIs, and/or other antiviral compounds.

As an example, the present invention provides a method for treating or preventing AIDS by administering to a patient in need of such therapy an effective amount of a BPH that inhibits phosphorolytic excision of the NRTI from replicating HIV DNA and an effective amount of an NRTI and optionally another antiviral compound. When administering the BPH to the patient, the BPH may be administered prior to, co-administered, concurrently administered with, and/or sequentially administered with, an NRTI and/or other antiviral compound.

The present invention also provides methods for sensitizing a subject with a retrovirus-related disorder to treatment with a NRTI or another antiviral compound by administering an effective amount of a BPH.

The therapeutic effect of NRTIs, such as Zidovudine (ZDV, AZT), Didanosine (ddl), Zalcitabine (ddC), Stavudine (d4T), abacavir, Emtriva, and tenofovir, can be potentiated in accordance with the methods of the present invention. The NRTIs, and optionally other antiviral compound(s), can be administered prior to, co-administered, concurrently administered, and/or sequentially administered with the BPH. As a result, the BPH and the NRTI (and/or other antiviral compound) may synergistically act to prevent or treat the retroviral-related disorder.

The present invention also provides methods for inhibiting retroviral replication, lowering viral load, treating a retrovirus-related disorder, or preventing a retrovirus-related disorder by administering a BPH in combination with a reduced dose of a NRTI and/or with a reduced dose of another antiviral compound. The composition comprising BPH is administered prior to, co-administered, or concurrently administered and/or sequentially administered with an NRTI and/or another antiviral compound.

The present invention also provides methods for inhibiting retroviral replication, lowering viral load, treating a retrovirus-related disorder, or preventing a retrovirus-related disorder by administering a BPH and/or a NRTI and/or another antiviral compound for shorter periods of time when compared to standard treatment times using the NRTI and/or other antiviral compound.

The present invention also provides methods for inhibiting retroviral replication, lowering viral load, treating a retrovirus-related disorder, preventing a retrovirus-related disorder by administering a BPH and/or a NRTI and/or another antiviral compound for fewer treatment cycles when compared to standard treatment regimens using the NRTI and/or other antiviral compound.

The present invention also provides methods for reducing the occurrence of secondary infections (e.g., pneumonia) in a subject with a retrovirus-related disorder comprising administering an effective amount of a BPH.

The present invention also provides methods for manufacturing and synthesizing a BPH.

The present invention also provides methods for identifying an agent that modulates NRTI excision by a biphosphonate-based compound. For example, the agent can be identified by introducing the agent in a mixture having a BPH, a NRTI (e.g., AZT) and a retrovirus, or portion thereof (e.g., a fragment of HIV that binds the BPH thereby forming a complex of the BPH and the HIV fragment that binds the BPH), and measuring a change in the efficacy of chain terminator excision when compared to control.

### Definitions

As used herein, the phrase “nucleoside reverse transcriptase inhibitor” (NRTI) refers to a nucleoside compound that inhibits the replication of retroviral DNA. NRTIs are analogs of dNTP substrates that lack a 3′-hydroxy but nevertheless are incorporated into a growing DNA chain by the viral reverse transcriptase (“RT”). Once a NRTI is added to the 3′ end of the growing DNA chain, viral DNA replication is inhibited.

As used herein, the phrase “antiviral compound” refers to a compound that inhibits infection of, or replication of, a virus in vitro and/or in vivo.

As used herein, the phrase “inhibiting replication” refers to inhibiting retroviral DNA synthesis.

As used herein, the term “variant” refers to any pharmaceutically acceptable derivative, analogue, or fragment of a BPH, NRTI, or antiviral compound described herein. A variant also encompasses one or more components of a multimer, multimers comprising an individual component, multimers comprising multiples of an individual component (e.g., multimers of a reference molecule), a chemical breakdown product, and a biological breakdown product. In particular, non-limiting embodiments, a BPH may be a “variant” relative to a reference BPH by virtue of alteration(s) in a portion of the reference BPH. For example, [4-chloro-(biphen-3-yl) aminomethane]-1,1-bisphosphonate is a variant of is [(biphen-3-yl)aminomethylene]-1,1-bisphosphonate (“218A”) in which one of the hydrogens on one of the aromatic rings of the biphenyl ring system has been replaced by a chlorine atom.

As used herein, the phrase “reduced dose” refers to a dose that is below the normally administered and/or recommended dose. The normally administered dose of an antiviral compound can be found in reference materials well known in the art such as, for example, the latest edition of the Physician's Desk Reference.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein:

FIG. 1 shows the structure of bisphosphonate 218A.

FIG. 2 is a graph showing the effect of 218A on the rate of RT-catalyzed ATP-dependent excision of terminating AZT in vitro.

FIGS. 3 shows a dose-response curve of the effect of 218A on the rate of RT-catalyzed ATP-dependent excision of terminating AZT in vitro.

FIG. 4 shows a table demonstrating the anti-viral activity of AZT alone and in combination with varying concentrations of 218A.

FIG. 5 shows the effect of various BPHs on RT-catalyzed DNA synthesis and on RT-catalyzed ATP-dependent excision of terminating AZT in vitro.

FIG. 6 shows the anti-viral activity of various BPHs alone and in combination with AZT.

### DETAILED DESCRIPTION OF THE INVENTION

The present invention provides compositions comprising a BPH, or a variant thereof, and methods for using such compositions for treating a retrovirus-related disorder, e.g., AIDS. Accordingly, a subject in need of such treatment is administered an effective amount of a composition comprising BPH. The subject may be a human or a non-human animal. The present invention is based, in part, on the discovery that bisphosphonates potently inhibited RT-catalyzed excision of chain-terminating NRTIs from the primer 3′ end of AZT-terminated DNA from a retrovirus in a cell-culture model. The present invention is based, in part, on the discovery that bisphosphonates potently inhibited RT-catalyzed excision of chain-terminating NRTIs from the primer 3′ end of AZT-terminated DNA in in vitro biochemical assays, and significantly potentiated the antiviral activity of AZT against thymidine analogue-associated mutations (“TAM”)-containing HIV-1 in a cell-culture model.

The present invention provides a composition comprising bisphosphonate (“BPH”). In particular, the present invention is related to the use of one or more BPHs in combination with one or more nucleoside reverse transcriptase inhibitors (“NRTIs”). Compositions of the present invention may also comprise one or more other antiviral compounds.

The bisphosphonate of the invention is capable of modulating NRTI excision. Preferably, the BPH of the present invention inhibits or slows the rate of chain terminator excision. While certain nonnucleoside RT inhibitors (NNRTI) such as UC781 have been shown to inhibit RT-catalyzed pyrophosphate-mediated excision of AZT 5′-monophosphate (“AZTMP”) (See Borkow et al. The thiocarboxanilide nonnucleoside inhibitor UC781 restores antiviral activity of 3′-deoxythymidine (AZT) against AZT-resistant human immunodeficiency virus type 1. Antimicrob Agents Chemother 1999; 43:259-263), no compounds of the bisphosphonate class of chemical structures have previously been shown to inhibit chain terminator excision. Furthermore, no class of chemical compound, including NRTIs, has previously been found to inhibit RT-catalyzed ATP-dependent excision of AZTMP. The mechanism of bisphosphonate inhibition of RT-catalyzed ATP-dependent or pyrophosphate-dependent excision of AZTMP may arise in part from binding of bisphosphonate to HIV-1 RT in a manner that prevents the binding of ATP and/or pyrophosphate, thereby inhibiting the nucleophilic attack of these excision substrates on the phosphodiester bond of the chain-terminating AZTMP. As such, the addition of BPH promotes the prolonged and continued use of NRTI drugs, such as AZT.

The present invention provides for compounds having the general formula (I): 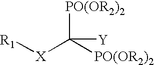
where X=NH, CH₂, CF₂, O, S
Y=H, OH
R₁=an aryl group, an arylalkyl group, an aryloxy group, an acyloxy group, an alkyl group, an alkenyl group, an alkynyl group, an alkoxy group, a haloalkyl group, a perfluoroalkyl group, a carbamoyloxy group, a cyanoalkyl group, an azidoalkyl group, a hydrazinoalkyl group, a hydroxyalkyl group, an alkoxyalkyl group, an aminoalkyl group, an alkylaminoalkyl group, a dialkylaminoalkyl group, an aryl aminoalkyl group, a diarylaminoalkyl group, an arylalkyl aminoalkyl group.
R₂=H, a methylene group, a carbonate group.

The present invention further provides for compounds having the general formula (II): 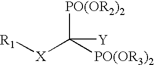
where X=NH, CH₂, CF₂, O, S
Y=H, OH
R₁=an aryl group, an arylalkyl group, an aryloxy group, an acyloxy group, an alkyl group, an alkenyl group, an alkynyl group, an alkoxy group, a haloalkyl group, a perfluoroalkyl group, a carbamoyloxy group, a cyanoalkyl group, an azidoalkyl group, a hydrazinoalkyl group, a hydroxyalkyl group, an alkoxyalkyl group, an aminoalkyl group, an alkylaminoalkyl group, a dialkylaminoalkyl group, an aryl aminoalkyl group, a diarylaminoalkyl group, an arylalkyl aminoalkyl group.

R₂=H, a methylene group, a carbonate group.

R₃=H, a methylene group, a carbonate group.

The terms “aryl”, “alkyl” and other groups refer generally to both unsubstituted and substituted groups unless specified to the contrary.

Unless otherwise specified, aryl groups include, but are not limited to, a phenyl group, benzyl group, biphenyl group, naphthyl group, quinolinyl group, isoquinolinyl group, indolyl group, pyrimidyl group, purinyl group, pyrrolyl group, furanyl group, imidazolyl group, oxazolyl group, thiazolyl group, pyrazolyl group, and carbazolyl group. The aryl groups may be both unsubstituted and substituted. Substituents may be at any position on the aryl group, and the aryl group may have one or more substituents. For example, aryl groups may preferably be substituted with a group or groups including, but not limited to, a halogen (fluorine, chlorine, bromine, iodine), a haloalkyl group (for example, perfluoroalkyl), a hydroxy group, an amino group (including, for example, free amino groups, alkylamino, dialkylamino groups and arylamino groups), an alkoxy group, an alkenyl group, an alkynyl group, an acyloxy group, and an aryl group. In the case of amino groups (—NR¹R^{m}), R¹ and R^{m} are preferably independently hydrogen, an acyl group, an alkyl group, or an aryl group. Acyl groups may preferably be substituted with (that is, Rⁱ is) an alkyl group, a haloalkyl group (for example, a perfluoroalkyl group), an alkoxy group, an amino group and a hydroxy group. Alkynyl groups and alkenyl groups may preferably be substituted with (that is, R^{j} and R^{k} are preferably) a group or groups including, but not limited to, an alkyl group, an alkoxyalkyl group, an amino alkyl group and an aryl group.

Unless otherwise specified, alkyl groups are hydrocarbon groups and are preferably C₁-C₁₀ alkyl groups (that is, having 1 to 10 carbon atoms), and more preferably C₂-C₆ alkyl groups, and can be branched or unbranched, acyclic or cyclic. The above definition of an alkyl group and other definitions apply also when the group is a substituent on another group (for example, an alkyl group as a substituent of an aryl group, an alkylamino group or a dialkylamino group).

The term “alkoxy” refers to —OR^{g}, wherein R^{g} is an alkyl group. The term “aryloxy” refers to —OR^{h}, wherein R^{h} is an aryl group. The term acyl refers to —C(O)Rⁱ. The term “alkenyl” refers to a straight or branched chain hydrocarbon group with at least one double bond, preferably with 2-10 carbon atoms, and more preferably with 2-6 carbon atoms (for example, —CH═CHR^{j} or —CH₂CH═CHR^{j}). The term “alkynyl” refers to a straight or branched chain hydrocarbon group with at least one triple bond, preferably with 2-10 carbon atoms, and more preferably with 2-6 carbon atoms (for example, —C≡CR^{k} or —CH₂—C≡CR^{k}). The terms “alkylene,” “alkenylene” and “alkynylene” refer to bivalent forms of alkyl, alkenyl and alkynyl groups, respectively.

The groups set forth above, can be substituted with a wide variety of substituents to synthesize bisphosphonate analogs. retaining activity. For example, alkyl groups may preferably be substituted with a group or groups including, but not limited to, a benzyl group, a phenyl group, an alkoxy group, a hydroxy group, an amino group (including, for example, free amino groups, alkylamino, dialkylamino groups and arylamino groups), an alkenyl group, an alkynyl group, a halogen (for example, perfluoroalkyl) and an acyloxy group. In the case of amino groups (—NR^{l}R^{m}), R^{l} and R^{m} are preferably independently hydrogen, an acyl group, an alkyl group, or an aryl group. Acyl groups may preferably be substituted with (that is, Rⁱ is) an alkyl group, a haloalkyl group (for example, a perfluoroalkyl group), an alkoxy group, an amino group and a hydroxy group. Alkynyl groups and alkenyl groups may preferably be substituted with (that is, R^{j} and R^{k} are preferably) a group or groups including, but not limited to, an alkyl group, an alkoxyalkyl group, an amino alkyl group and a benzyl group.

The term “acyloxy” as used herein refers to the group —OC(O)R^{g}.

The term “alkoxycarbonyloxy” as used herein refers to the group —OC(O)OR^{g}.

The term “carbamoyloxy” as used herein refers to the group —OC(O)NR^{l}R^{m}.

The R₂ and/or R₃ groups may be the same or different. The different R₂ and R₃ groups may result in a chiral molecule. The R₂ and/or R₃ groups may result in esters, hydrates, salt, or other pharmaceutically acceptable chemical groups. In specific non-limiting embodiments, R₂ and/or R₃ is/are pivaloyloxymethylene esters or isopropylcarbonates. The resulting compound may be a prodrug.

Bisphosphonates of the invention may include, but are not limited to, (4-amino-1-hydroxybutylidene)bis-phosphonate (“alendronate”), (Dichloromethylene)bis-phosphonate (“clodronate”), [1-hydroxy-3-(1-pyrrolidinyl)-propylidene]bis-phosphonate (“EB-1053”), (1-hydroxyethylidene)bis-phosphonate (“etidronate”), [1-hydroxy-3-(methyl pentyl amino)propylidene]bis-phosphonate (“ibandronate”), [Cycloheptylamino)-methylene]bis-phosphonate (“incadronate”), (6-amino-1-hydroxyhexylidene)bis-phosphonate (“neridronate”), [3-(dimethylamino)-1-hydroxypropylidene]bis-phosphonate (“olpadronate”), (3-amino-1-hydroxypropylidene)bis-phosphonate (“pamidronate”), [1-hydroxy-2-(3-pyridinyl)ethylene]bis-phosphonate (“risedronate”), [[(4-chlorophenyl)thiol]-methylene]bis-phosphonate (“tiludronate”), [1-hydroxy-2-imidazo-(1,2-a)pyridin-3-yl ethylidene]bis-phosphonate (“YH 529”), [1-hydroxy-2-(1 H-imidazol- 1-yl)ethylidene]bis-phosphonate (“zoledronate”).

Table 1 below provides a non-limiting list of BPH compounds of the present invention.

TABLE 1
AZT-excision activity of various BPH compounds

| | |
|---|---|
| | (I) |
| 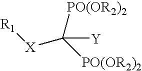 | |

| | | | | % Inhibition |
|---|---|---|---|---|
| Compound | | | | of AZT |
| Number | R₁ | X | Y | excision |
| 11A | 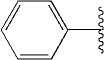 | CH₂ | OH | 30 |
| 23A | C₁₀H₂₁ | CH₂ | OH | 33 |
| 24A | 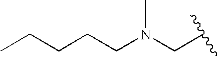 | CH₂ | OH | 38 |
| 30A | C₈H₁₇ | CH₂ | OH | 33 |
| 31A | cycloheptyl | NH | H | 38 |
| 56A | 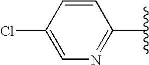 | NH | H | 31 |
| 79A | 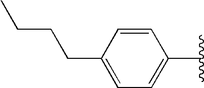 | NH | H | 26 |
| 80A | 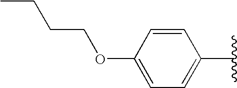 | NH | H | 30 |
| 96A | | 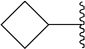 | OH | 40 |
| 202A | | 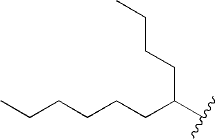 | OH | 30 |
| 203A | C₁₂H₂₅ | CH₂ | OH | 21 |
| 206A | 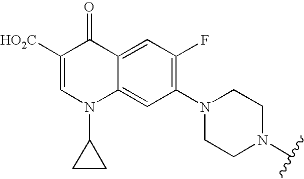 | CH₂ | H | 26 |
| 212A | 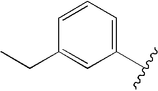 | NH | H | 65 |
| 213A | 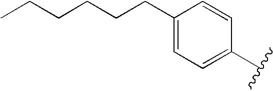 | NH | H | 32 |
| 214A | 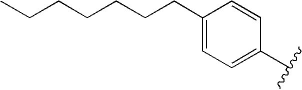 | NH | H | 27 |
| 215A | 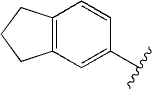 | NH | H | 75 |
| 217A | 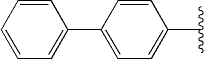 | NH | H | 20 |
| 218A | 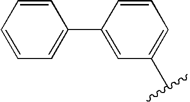 | NH | H | 72 |
| 222A | 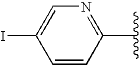 | NH | H | 40 |
| 227A | 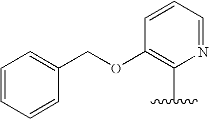 | NH | H | 36 |
| 228A | 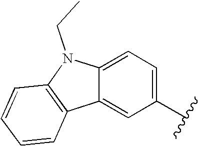 | NH | H | 60 |
| 232A | 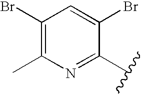 | NH | H | 56 |
| 237A | | 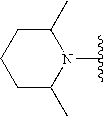 | H | 39 |
| 239A | 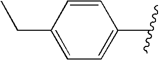 | NH | H | 24 |
| 248A | 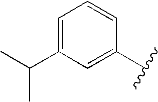 | NH | H | 59 |
| 249A | 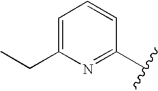 | NH | H | 25 |
| 250A | 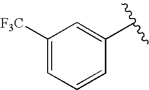 | CH₂ | OH | 68 |
| 251A | 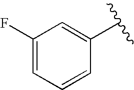 | CH₂ | OH | 68 |
| 253A | 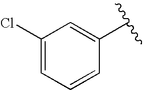 | CH₂ | OH | 70 |
| 255A | 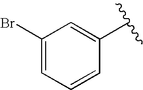 | CH₂ | OH | 76 |
| 256A | 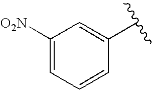 | CH₂ | OH | 66 |
| 258A | 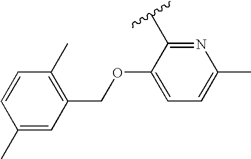 | NH | H | 23 |
| 261A | 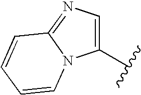 | CH₂ | OH | 25 |
| 262A | 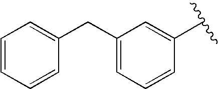 | NH | H | 66 |
| 263A | 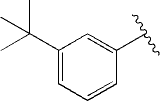 | NH | H | 62 |
| 264A | 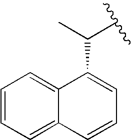 | NH | H | 34 |
| 266A | 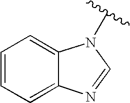 | CH₂ | OH | 25 |
| 267A | 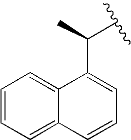 | NH | H | 61 |

Table 2 below provides a list of preferred variants of a BPH of the present invention.

TABLE 2
Preferred BPH variants

| Compound # | Structure | Estimated IC50(μM) |
|---|---|---|
| 342A | 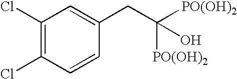 | 0.4 |
| 314A | 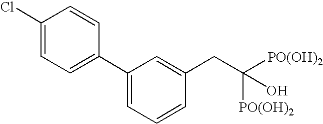 | 0.9 |
| 302A | 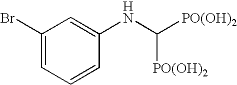 | 1.0 |
| 336A | 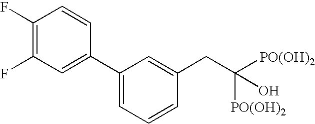 | 1.0 |
| 279A | 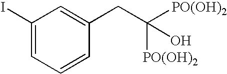 | 1.1 |
| 257A | 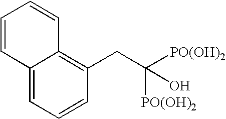 | 1.35 |
| 334A | 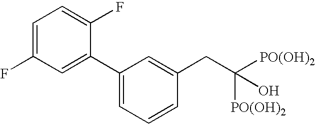 | 1.5 |
| 255A | 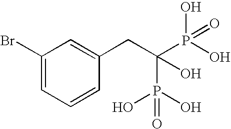 | 1.8 |
| 313A | 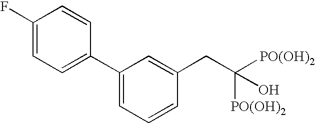 | 1.9 |
| 327A | 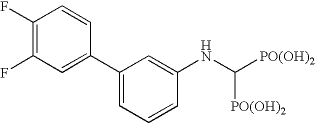 | 2.1 |
| 317A | 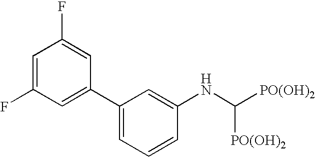 | 2.1 |
| 315A | 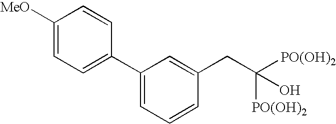 | 2.4 |
| 256A | 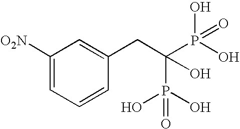 | 2.5 |
| 304A | 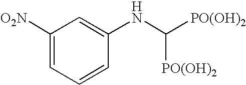 | 2.7 |
| 218A | 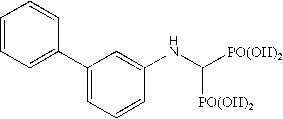 | 2.7 |
| 325A | 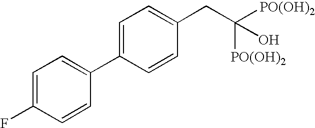 | 2.9 |
| 250A | 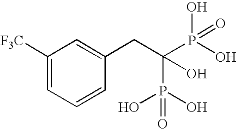 | 2.9 |
| 253A | 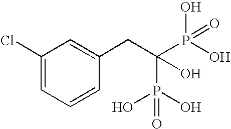 | 3.1 |
| 339A | 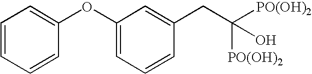 | 3.3 |
| 48B | 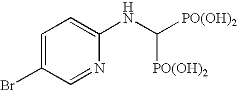 | 3.4 |
| 251A | 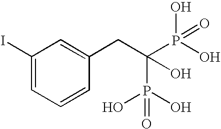 | 3.4 |
| 332A | 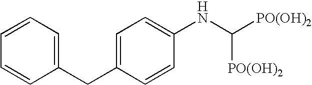 | 3.5 |
| 248A | 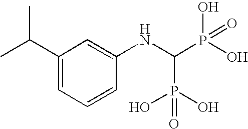 | 3.7 |
| 316A | 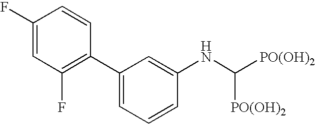 | 3.9 |
| 326A | 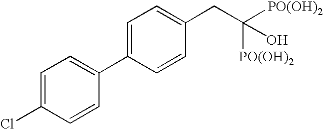 | 3.9 |
| 228A | 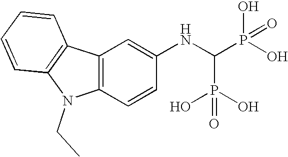 | 4.1 |
| 262A | 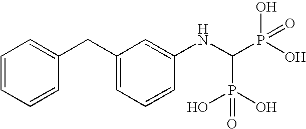 | 4.2 |
| 215A | 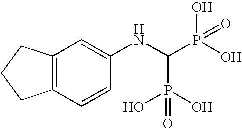 | 4.5 |
| 331A | 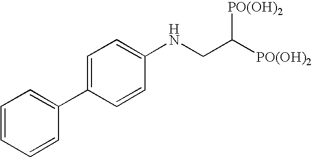 | 4.6 |
| 329A | 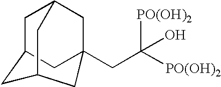 | 4.6 |
| 340A | 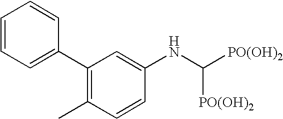 | 5.0 |
| 333A | 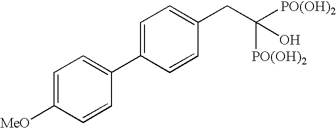 | 5.2 |
| 341A | 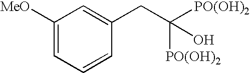 | 5.4 |
| 280A | 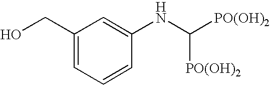 | 7.9 |
| 244A | 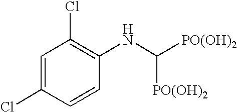 | 8.0 |
| 289A | 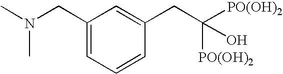 | 8.3 |
| 281A | 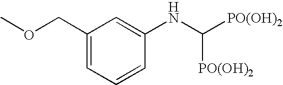 | 9.5 |
| 312A | 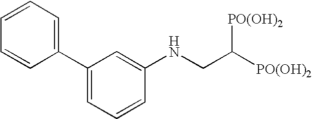 | 11.3 |
| 298A | 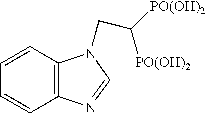 | 13.5 |
| 311A | 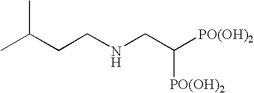 | 15.5 |

In a specific, non-limiting embodiment, a BPH comprises [(biphen-3-yl)aminomethylene]-1,1-bisphosphonate (hereinafter referred to as “218A”). In other non-limiting embodiments, the BPH comprises one or more variants of 218A. Under physiologic conditions, a 218A variant preferably inhibits the excision of a NRTI, by at least 25%, 50%, 60%, 70%, 80%, 90%, or 95% when compared to the inhibiting activity of 218A under similar conditions.

The novel BPH compounds of the present invention include, but are not limited to, Compound Nos. 79, 80, 96, 202, 206, 212, 213, 214, 215, 217, 218, 222, 227, 228, 232, 239, 248, 249, 250, 251, 255, 256, 258, 262, 263, 264, 266, 267, as listed on Table 1, and compounds 48B, 244A, 253A, 257A, 279A, 280A, 281A, 289A, 298A, 302A, 304A, 311A, 312A, 313A, 314A, 315A, 316A, 317A, 325A, 326A, 327A, 329A, 331A, 332A, 333A, 334A, 336A, 339A, 340A, 341A, 342A, as listed on Table 2.

The present invention provides a method for synthesizing the compounds of the present invention. In a non-limiting embodiment, the method includes synthesizing a BPH compound as taught in Example 1 below. Guidance may also be provided through methods of synthesizing bisphosphonates known to a person of ordinary skill in the art. (See, e.g., Soloducho et al. Patent PL93-298436 (1997). Preparation of novel derivatives of (aminomethylene)bis(phosphonic acid) as herbicides; Kieczykowski et al., J. Org. Chem., 60, 8310-8312 (1995). Preparation of (4-amino-1-hydroxybutylidene) bisphosphonic and sodium salt, MK-217 (alendronate sodium). An improved procedure for the preparation of 1-hydroxy-1,1-bisphosphonic acids.)

The practice of the present invention employs, unless otherwise indicated, conventional techniques of synthetic organic chemistry, protein chemistry, molecular biology, microbiology, and recombinant DNA technology, which are well within the skill of those in the art. Such techniques are explained fully in the literature. See, e.g., Scopes, R. K., Protein Purification Principles and Practices, 2d ed. (Springer-Verlag, 1987), Methods in Enzymology (S. Colowick and No. Kaplan, eds., Academic Press, Inc.), Sambrook et al., Molecular Cloning: A Laboratory Manual, 2d ed., Cold Spring Harbor Press, Cold Spring Harbor, N.Y., 1989, Handbook of Experimental Immunology, Vols. I-IV (D. M. Weir and C. C. Blackwell, eds., 1986, Blackwell Scientific Publications); House, Modem Synthetic Reactions, 2d ed., Benjamin/Cummings, Menlo Park, Calif., 1972.

The present invention also provides a composition comprising a substantially isolated complex of a BPH (or a variant thereof) and a bisphosphonate-recognition site. In a non-limiting embodiment, the complex further comprises a NRTI.

The present invention provides for screening assays using the compositions comprising a substantially isolated BPH and a bisphosphonate recognition site. Accordingly, in a non-limiting embodiment, the invention provides a method for identifying an agent that modulates bisphosphonate-mediated NRTI excision. The method includes contacting with a test compound a composition comprising a substantially isolated complex of a BPH (or a variant thereof) and a bisphosphonate-recognition site, and measuring a modulating effect, if any (e.g., determining the presence or absence of a modulating effect), of the test compound on bisphosphonate-mediated NRTI excision activity. Determination of a modulating effect on bisphosphonate-mediated NRTI excision would thereby indicate the presence of an agent that modulates NRTI excision by the BPH.

In a non-limiting embodiment of the present invention, the bisphosphonate-recognition site is a nucleic acid excision substrate. The nucleic acid excision substrate can be derived by, for example, annealing template nucleic acid to a labeled primer/template oligonucleotide, subjected to chain-termination using AZT 5′-monophosphate (“AZTMP”), as described in Example 5. The nucleic acid excision substrate can be used in a reverse transcription reaction to evaluate the ability of various test compounds to modulate reverse transcription.

The present invention provides a method for modulating NRTI excision in a cell. The method includes contacting the cell with an effective amount of one or more BPHs in combination with NRTI to reduce and/or inhibit NRTI chain termination excision. The BPH may be introduced prior to, overlapping with, concurrently, and/or after introduction of the AZT. In a further embodiment, the cell is infected with a NRTI-resistant retrovirus.

The present invention provides a method for prolonging the use and effectiveness of a NRTI for the treatment of NRTI-resistant antiviral disorder in a subject. The method includes administering to the subject an effective amount of one or more BPHs to reduce NRTI chain termination excision. In an embodiment of the invention, the antiviral disorder is HIV. In a further embodiment of the invention, the NRTI is AZT. The BPH may be introduced prior to, overlapping with, concurrently, and/or after introduction of the AZT.

The present invention provides a method for restoring the antiviral activity of NRTI drugs, such as AZT, in a cell infected with a NRTI-resistant retrovirus, such as HIV. The method comprises contacting the cell with an effective amount of one or more BPHs to inhibit viral replication of HIV. The BPH may be introduced prior to, overlapping with, concurrently, and/or after introduction of the AZT. In another non-limiting embodiment of the invention, the BPH inhibits reverse transcription.

The present invention also provides a method for inhibiting replication of a retrovirus in a cell. The method includes contacting the cell with a composition comprising an effective amount of one or more BPHs. In a non-limiting embodiment, the method further comprises contacting the cell with an effective amount of one or more NRTIs. In another non-limiting embodiment, the method further comprises contacting the cell with an effective amount of one or more other antiviral compounds. In another non-limiting embodiment, the NRTI and other antiviral compound may be contacted with the cell prior to, overlapping with, concurrently, and/or after introduction of the BPH.

Retroviral replication may be inhibited in a cell, tissue or organ culture, for example. The cells, tissues, or organ cultures may be of animal or human origin. In a non-limiting embodiment, the cells are derived from a patient infected with a retrovirus. The culture may comprise, without limitation, one or more cell types, one tissue type, or a combination of tissue types. The culture may be used as a model for retroviral replication, for example useful for testing the effectiveness of antiviral compounds, or to determine effective doses of antiviral compounds. The culture system may also be used to test the effectiveness of different combinations of antiviral compounds on viral replication. Cells infected with a retrovirus can be evaluated to determine the susceptibility of the tested retrovirus to the treatment methods of the invention. For example, human T-cells can be infected with HIV-1 in vitro, and viral replication measured in response to treatment with a BPH. The effect of a BPH or a variant thereof can be tested in this manner.

The present invention also provides a method for inhibiting retroviral replication in a subject. The method includes administering to the subject a composition of the present invention, i.e., a composition comprising an effective amount of one or more BPHs. In a non-limiting embodiment, the method further comprises administering an effective amount of one or more NRTIs and/or one or more other antiviral compounds. The effect of the method on retroviral replication in the subject can be determined using techniques for measuring retroviral replication that are known in the art. The NRTI and other antiviral compound may be administered to a subject prior to, overlapping with, concurrently, and/or after introduction of the BPH.

The present invention provides a method for reducing viral titer in a subject. The method includes administering to the subject a composition comprising an effective amount of one or more BPHs. The NRTI and other antiviral compound is administered to a subject prior to, overlapping with, concurrently, and/or after introduction of the BPH. The effect of the method on viral titer in the subject can be determined using techniques for monitoring viral titer that are known in the art. The BPH is administered prior to, co-administered, concurrently administered and/or sequentially administered with an NRTI and/or another antiviral compound.

The present invention also provides a method for treating a retrovirus-related disorder. The method includes administering to a subject in need of such treatment an effective amount of one or more BPHs. An effective amount is that amount sufficient to reduce or inhibit NRTI chain termination excision. In a preferred embodiment, the retrovirus-related disorder is AIDS. The NRTI and other antiviral compound may be administered to the subject prior to, overlapping with, concurrently, and/or after introduction of the BPH. The effect of the method on treating or ameliorating the retroviral disorder in the subject can be evaluated using clinical indicia practiced by the skilled artisan in the management of retrovirus-infected subjects.

The present invention also provides a method for preventing a retrovirus-related disorder. The method includes administering to a subject in need of such preventative treatment an effective amount of one or more BPHs. The NRTI and other antiviral compound may be administered to the subject prior to, overlapping with, concurrently, and/or after introduction of the BPH.

In a non-limiting embodiment, the retrovirus-related disorder is caused by infection with a virus such as HIV-1, HIV-2, HTLV-1 and HTLV-2, as well as variants of SIV. Non-retrovirus disorders include infection by the human hepadnavirus hepatitis B virus. For veterinary use, the retrovirus-related disorder is caused by infection with a virus such as SIV, EIAV, FLV, or FIV.

The present invention provides a method for preventing recurrence of a retrovirus-related disorder. The method includes administering to in a subject in need of such preventative treatment, an effective amount of one or more BPHs. The present invention also provides a method for reducing the rate of recurrence of a retrovirus-related disorder. The method includes administering to a subject in need of such preventative treatment an effective amount of one or more BPHs. The NRTI and other antiviral compound may be administered to the subject prior to, overlapping with, concurrently, and/or after introduction of the BPH.

In a particular embodiment, the present invention provides a method for treating AIDS. The method includes administering to a subject infected with HIV an effective amount of AZT in combination with an effective amount of a BPH that inhibits phosphorolytic excision of AZT from HIV DNA. When administering a BPH to the subject, the BPH is administered prior to, co-administered, concurrently administered, and/or sequentially administered with an NRTI and/or another antiviral compound. A typical regimen involving oral administration of AZT is typically about 100 mg every 4 hours or so. This corresponds to approximately 10 mg/kg for an individual weighing 60 kg.

The present invention also provides a method for sensitizing a subject infected with a retrovirus and/or having a retrovirus-related disorder to treatment with a NRTI or another antiviral compound. The method includes administering an effective amount of a BPH. When administering a BPH to the subject, the BPH is administered prior to, co-administered, concurrently administered, and/or sequentially administered with an NRTI. In a non-limiting embodiment, the BPH is administered prior to the administration of the NRTI, and optionally, another antiviral compound. As a result, the BPH and the NRTI (and/or other antiviral compound) may synergistically act to combat the disorder.

The therapeutic effect of NRTIs, including but not limited to Zidovudine (“ZDV”, “AZT”), Lamivudine (“3TC”), Didanosine (“ddI”), Zalcitabine (“ddC”), Stavudine (“d4T”), abacavir, tenofovir and Emtriva, can be potentiated in accordance with the methods of the present invention. The NRTIs, and optionally other antiviral compound(s), can be administered prior to, co-administered, concurrently administered, and/or sequentially administered with an NRTI and/or another antiviral compound.

In a non-limiting embodiment, the BPH and NRTI are administered concurrently. In a non-limiting embodiment, the BPH and NRTI are administered in a single formulation. In a non-limiting embodiment, the BPH and NRTI are administered in separate formulations. In another embodiment, the method further comprises administering the BPH and NRTI by different modes of administration.

Where one or more NRTIs are administered in addition to the one or more BPHs, the NRTI can include, without limitation, AZT, 3TC, ddl, ddC, d4T, abacavir, tenofovir and/or Emtriva. The NRTIs may be administered individually or in combinations of each other. Such combinations include, without limitations, Stavudine (“d4T”) and Didanosine (“ddl”), Stavudine (“d4T”) and Lamivudine (“3TC”), zidovudine (“AZT”) and Didanosine (“ddl”), zidovudine (“AZT”) and Zalcitabine (“ddC”), Zidovudine (“AZT”) and Lamivudine (“3TC”), and triple combinations such as, but not limited to, Didanosine (“ddI”), Zalcitabine (“ddC”), and Stavudine (“d4T”), and Zidovudine (“AZT”), Lamivudine (“3TC”) and abacavir.

Where one or more other antiviral compounds are administered in addition to the one or more BPHs, the other antiviral compound may include, without limitation, a NRTI (e.g. zidovudine (“AZT”), didanosine (“DDI”), dideoxycytidine (“DDC”), d4T, ribavirin, 3TC, Pyridnone, Abacavir”), a non-nucleoside reverse transcriptase inhibitor (“NNRTI”) (e.g., nevirapin, Delaviridine, Efavirenz), protease inhibitors (e.g., Saquinavir, Ritonavir, Indinavir, Telinavir, Amprenavir and Nelfinavir), integrase inhibitors, (e.g., AR177), a gene therapy inhibitor targeting a regulatory protein of HIV replication such as inhibitors of the rev protein (e.g., Rev M10), nucleocapsid inhibitor (e.g., DIBAs), inhibitor targeting the specific messenger RNA transcripts of all known HIVs (e.g., GEM92 and GPI-2A), inhibitor of the family of modulators of cellular dNTP (e.g., hydroxyurea), cytokine inhibitors (e.g. TNF), inhibitor of cellular entry of HIV (e.g. T20, “Fuzeon” or “enfuvirtide”, T1249, and SPC-3), and agents constituting therapeutic classes used in vaccines, (e.g. HIVAC-le, ALVAC, RG-8394), amphotericin B (a lipid-binding molecule) and castanospermine (an inhibitor of glycoprotein processing), 2-deoxy-D-glucose (“2-dGlc”), deoxynojirimycin, acycloguanosine, rifampicin (“rifadin”), adamantidine, rifabutine, ganciclovir (“DHPG”), fluoroiodoaracytosine, idoxurine, trifluorothymidine, adenine arabinoside (“ara-A”), ara-AMP, bromovinyldeoxyuridine, bromovinylarauracil (“BV-araU b” or 1-beta-D-arabinofuranoside-E-5-[2-bromovinyl]uracil), rimantadine, arildone, diarylamidine, (S)-(p-nitrobenzyl-)6-thioinosine and phosphonoformate, inhibitors of pyrophosphorolysis and/or inhibitors of ribonucleotide-dependent phosphorolysis.

In particular embodiments, the other antiviral compound may include a nucleoside derivative such as, without limitation, 2′,3′-dideoxyadenosine (“ddA”); 2′,3′-dideoxyguanosine (“ddG”); 2′,3′-dideoxyinosine (“ddl”); 2′,3′-dideoxycytidine (“ddC”); 2′,3′-dideoxythymidine (“ddT”); 2′,3′-dideoxy-dideoxythymidine (“d4T”) and 3′-azido-2′,3′-dideoxythymidine (“AZT”). Alternatively, halogenated nucleoside derivatives may be used, preferably 2′,3′-dideoxy-2′-fluoronucleosides including, but not limited to, 2′,3′-dideoxy-2′-fluoroadenosine; 2′,3′-dideoxy-2′-fluoroinosine; 2′,3′-dideoxy-2′-fluorothymidine; 2′,3′-dideoxy-2′-fluorocytosine; and 2′,3′-dideoxy-2′,3′-didehydro-2′-fluoronucleosides including, but not limited to 2′,3′-dideoxy-2′,3′-didehydro-2′-fluorothymidine (“Fd4T”). Preferably, the 2′,3′-dideoxy-2′-fluoronucleosides of the invention are those in which the fluorine linkage is in the beta configuration, including, but not limited to, 2′3′-dideoxy-2′-beta-fluoroadenosine (“F-ddA”), 2′,3′-dideoxy-2′-beta-fluoroinosine (“F-ddI”), and 2′,3′-dideoxy-2′-beta-fluorocytosine (“F-ddC”), uridine phosphorylase inhibitors, including but not limited to acyclouridine compounds, including benzylacyclouridine (“BAU”), benzyloxybenzylacyclouridine (“BBAU”); aminomethyl-benzylacyclouridine (“AMBAU”); aminomethyl-benzyloxybenzylacyclouridine (“AMB-BAU”); hydroxymethyl-benzylacyclouridine (“HMBAU”); and hydroxymethyl-benzyloxyben-zylacyclouridine (“HMBBAU”).

In particular embodiments, the other antiviral compound includes a cytokine or cytokine inhibitor such as, without limitation, rIFN-alpha, rIFN-beta, rIFN-gamma, inhibitors of TNF-alpha, and MNX-160.

The BPH(s), NRTI(s), and/or other antiviral agent(s) can also be administered in combination with one or more antibiotics, antifungal agents, immunotherapeutics, anti-angiogenic agents, vaccines, hormones, or other pharmaceutical agents effective for treating a retrovirus-related disorder.

Combination therapy comprising BPH, NRTI, and/or other antiviral compounds may sensitize the viral infection to treatment comprising administering additional antiviral compounds. Accordingly, the present invention contemplates combination therapies for preventing, treating, and/or preventing recurrence of a retrovirus-related disorder comprising administering an effective amount of a BPH prior to, subsequently, or concurrently with, a reduced dose of an antiviral compound. For example, initial treatment with a BPH may increase the sensitivity of a subject to subsequent challenge with a dose of antiviral compound. This dose is near, or below, the low range of standard dosages for the antiviral compound when the compound is administered alone, or in the absence of BPH.

Accordingly, in one embodiment, the additional antiviral compound comprises zidovudine at a concentration of about 400, 500, 600, or 700 mg/day.

In another embodiment, the additional antiviral compound comprises zalcitabine at a concentration of about 1, 1.75, 2.25, or 2.75 mg/day.

In another embodiment, the additional antiviral compound comprises stavudine at a concentration of about 70, 80, 90 or 100 mg/day.

In another embodiment, the additional antiviral compound comprises indinavir at a concentration of about 1800, 2000, 2200, 2400, or 2600 mg/day.

In another embodiment, the additional antiviral compound comprises ritonavir at a concentration of about 800, 900, 1000, 1100, 1200, or 1300 mg/day.

In another embodiment, the additional antiviral compound comprises saquinavir at a concentration of about 3000, 3200, 3400, 3600, or 3800 mg/day.

The present invention also provides methods for inhibiting retroviral replication, treating a retrovirus-related disorder, or preventing a retrovirus-related disorder. These methods include administering a BPH in combination with a reduced dose of NRTI and/or with a reduced dose of another antiviral compound. As such, clinically significant efficacy and/or reduced toxicity may be observed using methods known to the skilled artisan when using such combination therapies. For example, adverse reactions such as neuropathy and pancreatitis, which are sometimes observed when using certain NRTI combination therapies, may be lessened or avoided. The BPH can be administered prior to, co-administered with, concurrently administered with, and/or sequentially administered with, the NRTI and/or another antiviral compound.

The effective dose of the BPH to be administered during a cycle varies according to the mode of administration. Moreover, the effective dose of a specific BPH may depend on additional factors, including the type of retroviral disorder, the stage of the disease, the toxicity of the BPH to the patient, as well as the age, weight, and health of the patient. Taking this into account, one of ordinary skill in the art can determine the effective dose of a given BPH.

In one embodiment, the effective dose of the BPH results in a plasma concentration of 0.1-1000, 0.1-500, 0.1-100, 0.5-50, 0.5-10, 0.5-5, or 0.1-1 micrograms/liter. In a particular, non-limiting embodiment, the effective dose of the BPH is 0.1 to 50 micrograms/liter.

In one embodiment, the effective dose by of the BPH may range from about 5 micrograms to 2 grams/kg/day.

In another embodiment, the effective dose of the BPH may range from about 100 to 50,000 micrograms/kg/month.

In another particular embodiment, the effective dose of the BPH is between about 100 and 500 micrograms/kg/day, wherein the patient is administered a single dose per day. The single dose is administered every other day for approximately 5, 7, 9, 11, 13, 15, 17, 19, 21, 23, 25, 27, 29, or 31 consecutive days. After this cycle, a subsequent cycle may begin approximately 1, 2, 3, 4, 5, 6, 7, 8, 9, 10, 11, or 12 weeks later. The treatment regime may include 1, 2, 3, 4, 5, or 6 cycles, each cycle being spaced apart by approximately 1, 2, 3, 4, 5, 6,7,8,9, 10, 1, or 12weeks.

In a particular non-limiting embodiment, the effective dose of the BPH is between about 500 and 5000 micrograms/kg/month, wherein the patient is administered a single dose per day. The single dose is administered approximately every month for approximately 1, 2, 3, 4, 5, or 6 consecutive months. After this cycle, a subsequent cycle may begin approximately 1, 2, 4, 6, or 12 months later. The treatment regime may include 1, 2, 3, 4, 5, or 6 cycles, each cycle being spaced apart by approximately 1, 2, 4, 6, or 12 months.

In a specific non-limiting embodiment, a preferred BPH compound is administered at a dose to bring the plasma concentration of administered compound to 0.1 to 50 micrograms/liter, wherein the patient is administered a single dose per day. The single dose is administered every day for approximately five consecutive days. After this cycle, a subsequent cycle may begin approximately one month later, preferably one month from the first day of the first cycle. The treatment regime may include three cycles, each cycle being spaced apart by approximately one treatment-free week.

The effective dose of another antiviral compound to be administered during a cycle in combination with a BPH may also vary according to the mode of administration. Typically, antiviral compounds are administered systemically. Standard dosage and treatment regimens are known in the art (see, e.g., the latest editions of the Merck Index and the Physician's Desk Reference).

Indeed, treatment of a subject involving administering an effective amount of a BPH to the subject in need of such treatment may result in another antiviral compound exhibiting clinically significant efficacy even at reduced doses. The observed efficacy of the reduced dose of the other antiviral compound may not be realized absent administration in combination with a BPH. Thus, in accordance with the present invention, combination therapies comprising a BPH and one or more other antiviral compounds may reduce toxicity (i.e., side effects) of the overall treatment for the retrovirus-related disorder. For example, reduced toxicity, when compared to a monotherapy or another combination therapy, may be observed when delivering a reduced dose of the BPH and/or NRTI, and/or when reducing the duration of a cycle (i.e., the period of a single administration or the period of a series of such administrations), and/or when reducing the number of cycles.

Combination therapy may thus increase the sensitivity of the retrovirus to the administered BPH, and/or NRTI, and/or additional antiviral compounds. In this manner, shorter treatment times and/or fewer treatment cycles may nevertheless be clinically effective with fewer toxic events. Combination therapy involving administering a BPH to a patient in need of such treatment may permit relatively short treatment times when compared to the duration or number of cycles of standard treatment regimens. Accordingly, the present invention provides methods for treating a retrovirus-related disorder comprising administering one or more other antiviral compounds for relatively short duration and/or in fewer treatment cycles.

Accordingly, the invention provides a method for treating or preventing a retrovirus-related disorder comprising administering to a patient in need thereof an effective amount of one or more BPHs, and/or one or more NRTIs, and/or one or more other antiviral compounds for a short treatment cycle, ranging from approximately 1 to 14 days. The cycle duration may vary according to the specific NRTI or other antiviral compound in use.

In particular embodiments, continuous or discontinuous administration may be used, or daily doses may be divided into several partial administrations. An appropriate duration of a reduced cycle for a particular NRTI or other antiviral compound will be appreciated by the skilled artisan, as optimal treatment schedules for each NRTI or other antiviral compound are continually assessed. Specific guidelines for the skilled artisan are known in the art. (See, e.g., Dybul et al. and the Panel on Clinical Practices for Treatment of HIV. Guidelines for using antiretroviral agents among HIV-infected adults and adolescents. MMWR 2002; 51(No. RR-7): 1-55.; Dybul et al. (Editors) and the Panel on Clinical Practices for Treatment of HIV. Guidelines for using antiretroviral agents among HIV-infected adults and adolescents. Ann Intern Med 2002; 137(S):381-433.)

The present invention also provides a method for inhibiting retroviral replication, treating a retrovirus-related disorder, or preventing a retrovirus-related disorder, by administering one or more BPHs and/or one or more NRTIs and/or one or more other antiviral compounds for shorter periods of time when compared to accepted or standard treatment times using the NRTI and/or other antiviral compounds.

Alternatively, in certain situations, longer treatment cycles may be desired. Accordingly, the cycle duration may range from approximately 45 to 90 days. The duration of each cycle may vary according to the particular NRTI and/or other antiviral compound used.

The present invention also provides methods for inhibiting retroviral replication, treating a retrovirus related disorder, or preventing a retrovirus related disorder by administering one or more BPHs in fewer treatment cycles when compared to standard dosages or treatment times for the NRTI or other antiviral compound.

The present invention contemplates at least one cycle, preferably more than one cycle during which a combination therapy is administered. An appropriate total number of cycles, and the interval between cycles, will be appreciated by the skilled artisan. The number of cycles may be approximately 1-20 cycles. The interval between cycles may be approximately 1-30 days. Optimal treatment scheduling for each BPH, NRTI, and/or other antiviral compound may be determined by the skilled artisan through continued assessment of in vitro and/or in vivo trials.

In a non-limiting embodiment, a BPH and/or NRTI, and/or other antiviral compound is administered at high doses for short periods when compared to accepted or standard dosages and treatment times.

The BPH and other antiviral compounds of the invention may be effective over a wide ratio, the ratio ranging from approximately 1:1 to 1000:1. In a preferred embodiment, the ratio of the BPH to the other antiviral compound is between 1:1 to 50:1. Ratios may be adjusted accordingly when a further antiviral compound as administered.

The invention also provides methods for reducing the occurrence of secondary infections in a subject with a retrovirus-related disorder. The method includes administering an effective amount of a BPH.

In a preferred embodiment, the present invention provides a method for treating and/or ameliorating the clinical condition of patients suffering from AIDS. Accordingly, in one embodiment, the invention provides a method for inhibiting the proliferation of HIV. In another embodiment, the invention provides a method for preventing HIV replication. In another embodiment, the invention provides a method for lowering the HIV titer in the AIDS patient. In another embodiment, the invention provides a method for prolonging the disease-free interval following antiviral treatment.

Clinical outcomes of antiviral treatments using a BPH of the invention are readily discernible by the skilled artisan, i.e., the physician. Standard medical tests that evaluate clinical markers of a retrovirus-related disorder may be reliable indicators of the treatment's efficacy. Such tests may include, without limitation, physical examination, pain assessment, blood or serum chemistry, urinalysis, T-cell counts, viral titer, recordation of adverse events, assessment of infectious episodes, and pharmacokinetic analysis. Furthermore, synergistic effects of a combination therapy comprising a BPH, and a NRTI and/or other antiviral compound may be determined by studies of patients undergoing monotherapy compared to patients undergoing the combination therapy.

Pharmaceutical Compositions

A BPH according to the invention may be formulated in a pharmaceutical composition. Pharmaceutical compositions include water, alcohols, polyols, glycerin and vegetable oils, lyophilized powders, aqueous or non-aqueous sterile injectable solutions or suspensions, antioxidants, buffers, bacteriostats and solutes that render the compositions substantially isotonic. Extemporaneous injection solutions and suspensions may be prepared from sterile powders, granules and tablets. A BPH may be supplied, for example, as a lyophilized powder that can be reconstituted with an aqueous injectable solution prior to administration to a subject.

Pharmaceutical compositions of the invention can comprise a pharmaceutically acceptable carrier. Suitable pharmaceutically acceptable carriers include essentially chemically inert and nontoxic compositions that do not interfere with the effectiveness of the biological activity of the pharmaceutical composition.

As used herein, the phrase “pharmaceutically acceptable” refers to general clinical use and/or approved by a regulatory agency of a state or the Federal government, listed in the United States Pharmacopoeia, or generally accepted by the skilled artisan. Examples of suitable pharmaceutical carriers include, without limitation, water, saline solutions, glycerol solutions, ethanol, N-(1(2,3-dioleyloxy)propyl)N,N,N-trimethylammonium chloride (DOTMA), diolesylphosphotidyl-ethanolamine (DOPE), and liposomes. In a non-limiting embodiment, a pharmaceutical composition of the invention comprises a therapeutically effective amount of a BPH and a NRTI and/or other antiviral compound, together with a suitable amount of a pharmaceutically acceptable carrier so as to yield a form for intravenous administration to a subject.

The pharmaceutical composition may be in the form of a pharmaceutically acceptable salt which includes, without limitation, those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The present invention provides for methods of preparing the compositions of the invention. Various BPH compounds may be synthesized as discussed in Example 1 below.

Modes of Administration

Preferably, a BPH is formulated for systemic administration. Techniques for formulation and administration may be found, for example, in “Remington's Pharmaceutical Sciences,” Mack Publishing Co., Easton, Pa. Suitable routes may include, without limitation, oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections. Most preferably, administration of a BPH is intravenous. For injection, BPH may be formulated in an aqueous solution, preferably in physiologically compatible buffers such as for example Hanks' solution, Ringer's solution, or physiological saline buffer.

For such transmucosal administration, penetrants appropriate to the barrier to be permeated are used in the formulation. Such penetrants are generally known in the art.

In preferred, non-limiting embodiments, the BPH is administered orally.

Pharmaceutical Kits

The present invention also provides a pharmaceutical kit comprising an effective amount of one or more BPHs. In a non-limiting embodiment, the kit comprises, in at least one container, an effective amount of a BPH, an effective amount of a NRTI, and optionally an effective amount of one or more other antiviral compounds.

The present invention will be better understood by the following exemplary teachings. The examples set forth herein do not and are not intended to limit in any manner the present invention.

### EXAMPLES

### Example 1

### Synthesis of Various BPH Compounds

### Example 1.1

### Copound No. 218A

A mixture of 3-phenylaniline (2 g, 11.8 mmol), triethyl orthoformate (2.4 mL, 14.2 mmol), and diethyl phosphite (6.1 mL, 47.3 mmol) was heated at 140° C. under N₂ for 16 h.¹ The resulting oil was subjected to column chromatography (silica gel) with ethyl acetate and methanol (20:1, v/v) as the eluent to give the tetraethyl ester of 218A, which was subsequently dissolved in dry acetonitrile (15 mL) and treated with bromotrimethylsilane (7.5 mL) for 12 h. Upon removal of the solvent, the residue was treated with ethanol and water (20 mL, 1:1) to give a white precipitate, which was filtered and washed with ethanol to give 218A as a white powder (1.8 g, 40% overall yield.)

### Example 1.2

### Compound No. 255A

A mixture of 3-bromophenylacetic acid (1 g, 4.7 mmol), phosphorous acid (0.38 g, 4.7 mmol), and methanesulfonic acid (2 mL) was heated to 65° C. and phosphorus trichloride (0.85 mL, 9.8 mmol) was added dropwise under N₂.² The reaction mixture was stirred at the same temperature for 18 h. After cooling, 7 mL of water was added and the mixture was refluxed for 5 h. The pH of the resulting solution was adjusted to 4 by adding saturated NaOH solution and the monosodium salt of 255A precipitated and was collected by filtration (1.2 g, 67% overall yield).

### Example 1.3

### Compound No. 291A

To a suspension of NaH (26.4 mg, 1.1 mmol) in dry THF (5 mL) was added tetraethyl methylenediphosphonate (288 mg, 1 mmol). After 20 min, 3-phenylbenzyl bromide (247 mg, 1 mmol) was added to the above solution and the reaction mixture was stirred at room temperature for 12 h. After addition of saturated NH₄Cl solution, the product was extracted with ethyl acetate and purified by column chromatography (silica gel) with ethyl acetate and methanol (20 : 1, v/v) as the eluent to give the tetraethyl ester of 291A, which was subsequently dissolved in dry acetonitrile (2 mL) and treated with bromotrimethylsilane (0.7 mL) for 12 h. Upon removal of the solvent, the residue was treated with ethanol and water (5 mL, 1:1) for 1 h. The solvent was evaporated and the residue was dissolved in water (2 mL). Saturated NaOH solution was added to adjust the pH to 10. Ethanol was then added to give, after filtration, the tetrasodium salt of 291A as a white powder (204 mg, 46% overall yield).

### Example 1.4

### Compound No. 308A

A mixture of 2,4-difluorophenylboronic acid (0.75 g, 4.7 mmol), methyl 3-bromophenylacetate (0.9 g, 3.9 mmol), Pd(PPh₃)₄ (70 mg), and K₂CO₃ (0.85 g) in toluene (10 mL) and water (3 mL) was refluxed for 12 h. After cooling, the mixture was partitioned between ethyl acetate and brine. The organic layer was concentrated and subjected to column chromatography (silica gel) with hexane and ether (6:1, v/v) as the eluent to give methyl 3-(2,4-difluorophenyl)phenylacetate, which was then hydrolyzed with KOH (3 equiv.) in ethanol and water to give the corresponding acid. This was subsequently treated with oxalyl chloride (5 equiv.) in benzene in the presence of a catalytic amount of DMF, for 1 h. Upon removal of the solvent, the resulting acid chloride intermediate was dissolved in dry THF and reacted with 2 equiv. of tris(trimethylsilyl) phosphite, for 1 h.³ After evaporating the solvent, the residue was treated with methanol and water (10 mL, 1:1). Methanol was removed in vacuo and 308A was obtained by filtration as a white powder (0.67 g, 42% overall yield).

### Example 1.5

### Compound No. 300A

A mixture of 3-phenylpyridine (0.75 g, 5.2 mmol) and bromoacetic acid (0.72 g, 5.2 mmol) in ethyl acetate (5 mL) was stirred for 2 d and the precipitate was filtered. The resulting white powder (1.2 g), phosphorous acid (1.6 g), and phosphorus oxychloride (1.5 mL) in toluene (5 mL) was heated to 80° C. for 4 h with stirring. After cooling, the organic layer was decanted away and the viscous residue was dissolved in 5 mL of 6 N HCl and refluxed for 3 h. 2-Propanol was added to precipitate the product, which, after filtration, was redissolved in water (5 mL). The pH of the solution was adjusted to 8 by adding NaOH solution and ethanol was added to precipitate the product. The disodium salt of 300 A was obtained by filtration as a white powder (0.68 g, 30% overall yield).

### Example 2

### BPH Compound Inhibits of ATP-Mediated Phosphorolytic Excision of AZT Catalyzed by HIV-1 Reverse Transcriptase

### Example 2.1

### Methods

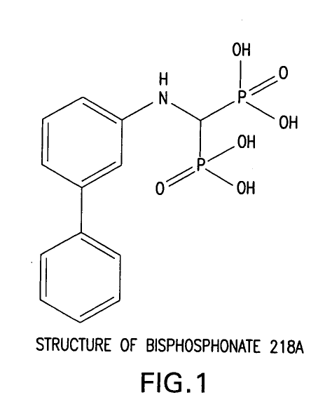

The effect of bisphosphonate, 218A shown in FIG. 1, on ATP-mediated excision of AZT was examined in vitro. An 18 nucleotide DNA oligonucleotide (“primer DNA”) of sequence 5′-GTCCCTGTTCGGGCGCCA-3′ was 5′ end labeled using [y³²p]-ATP and T4 polynucleotide kinase. The labeled primer DNA was purified by polyacrylamide gel electrophoresis, and then annealed to a synthetic DNA oligonucleotide (“template DNA”) of the sequence 5′-CTCAGACCCTTTTAGTCAGAATGG AAAATCTCTAGCAGTGGCGCCCGAACAGGGACA-3′, to form “template/primer DNA”. Template/primer DNA was then chain-terminated with AZTMP by incubating for 30 minutes at 37° C. with HIV-1 reverse transcriptase in 50 mM Tris-HCl, pH 7.8, containing 60 mM KCl, 10 mM MgCl₂, and 100 μM AZTTP. The total incubation sample was then dried under reduced pressure and the [³²P]-labeled, 3′-AZTMP-terminated primer DNA (now 19 nucleotides in length) was purified by polyacrylamide gel electrophoresis. The purified [³²P]-labeled, 3′-AZTMP-terminated primer DNA was annealed to template DNA to form the nucleic acid excision substrate (termed “AZTMP-T/P”).

AZTMP-T/P was mixed with a 3-fold molar excess of TAM-mutant HIV-1 reverse transcriptase in 50 mM Tris, pH 8.1, containing 60 mM KCl and 10 mM MgCl₂ and incubated for 5 minutes at 37° C. to allow formation of the RT-AZTMP-T/P binary complex. The bisphosphonate 218A was then added to the indicated final concentration, and reactions were initiated by the addition of ATP to 3 mM final concentration. At various times thereafter, aliquots were removed and quenched by dilution into sequencing gel loading buffer (98% deionized formamide, 10 mM EDTA, and 1 mg/mL each of bromophenol blue and xylene cyanol). Samples were heated at 100° C. for 5 minutes then subjected to polyacylamide gel electrophoresis using 16% polyacrylamide/7 M urea gels. Electrophoretically resolved products were visualized and quantified by phosphorimaging analysis. The figure shows the loss of the 19 nucleotide [³²P]-labeled, 3′-AZTMP-terminated primer DNA as a function of time of reaction in the presence of 3 mM ATP and 0 (O), 5μM 218A (▾) and 20μM 218A (●). The values indicated on the graph (k) are the calculated first order rate constants for RT-catalyzed ATP-dependent removal or excision of terminating AZTMP.

### Example 2.2

### The Rate of Excision is Significantly Decreased in the Presence of 218A

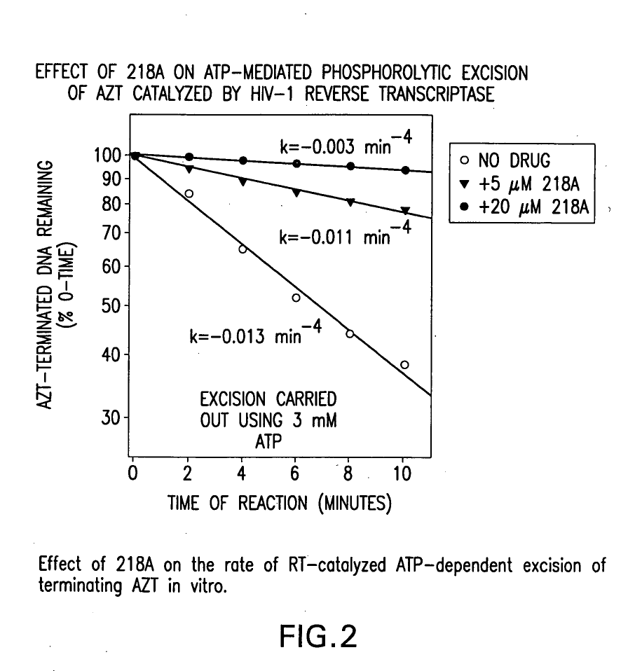

Excision of terminating AZT was measured over 10 minutes. In the absence of bisphosphonate, 65% of the AZT-terminated DNA had undergone excision after 10 min (k=−0.043/min) (FIG. 2). In the presence of 5 micromolar bisphosphonate only 15% of the AZT-terminated DNA had undergone excision (k=−0.011/min). In the presence of 20 micromolar 218A, less than 10% of the AZT-terminated DNA had undergone excision after 10 minutes (k=−0.003/min). The BPH compound, 218A, inhibited the rate of excision of AZT-terminated DNA in a dose dependent manner.

### Example 3. Concentration dependence of 218A inhibition of ATP-dependent phosphorolytic excision of AZT catalyzed by HIV reverse transcriptase

Reactions were carried out and first order rate constants for the excision of 3′-terminating AZTMP were calculated as described in Example 2.

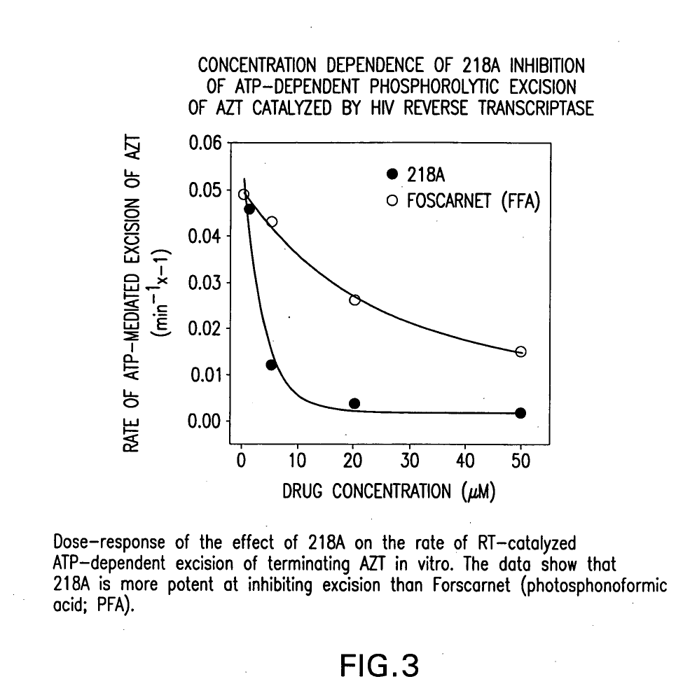

AZT-terminated DNA was incubated with increasing concentration of 218A or Foscamet, phosphonoformic acid. The 218A bisphosphonate showed a decrease in ATP-mediated excision of AZT with increasing concentration (FIG. 3). Increasing concentrations of Foscarnet did not inhibit ATP-mediated excision of AZT as well as 218A.

### Example 4

### BPH Compound 218A Increases the Sensitivity of AZT Treatment

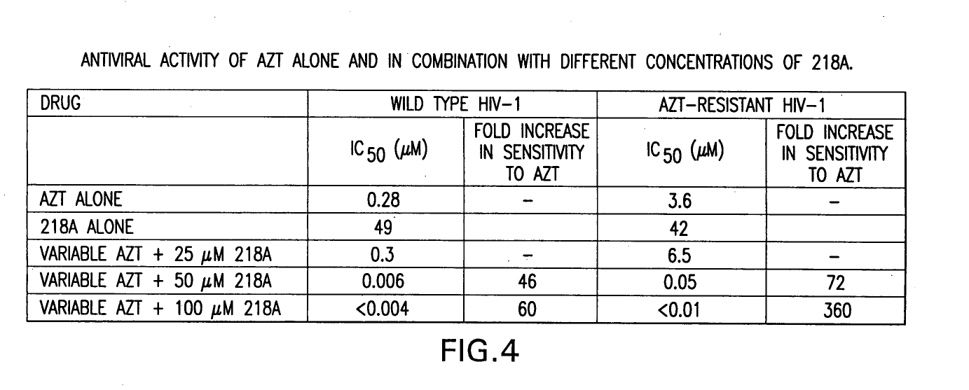

The antiviral activity of 218A was tested on both wild type and AZT-resistant HIV-1. 218A alone did not show antiviral activity in either wild type or AZT-resistant HIV-1 (FIG. 4). However, AZT in combination with concentrations of 218A up to 100 micromolar produced a 60 fold increase in sensitivity of wild type HIV-1 to AZT. The same concentration of 218A on AZT-resistant HIV-1 resulted in a 360 fold increase in sensitivity to AZT. Therefore, 218A is able to restore AZT-resistance to an AZT-resistant strain of virus. (FIG. 4).

218A alone is only weakly active against replication of wild type and AZT-resistant HIV-1 in P4/R5 cells (EC₅₀≈50 μM), and importantly shows no cytotoxicity at the highest tested concentration of 100 μM. When 218A and AZT are added in combination, marked enhancements of the antiviral effect is apparent. As an example, AZT is only marginally active against AZT-resistant HIV (EC₅₀≈1.2 μM compared to 0.14 μM against wild type virus). In the presence of 50 μM 218A, the activity of AZT against AZT-resistant HIV is increased over 100-fold (EC₅₀>0.025 μM), suggesting that 218A is able to restore activity of AZT against AZT-resistant virus. This “restorative” property of 218A is dose-dependent.

### Example 5

### Method for Screening of Bisphosphonate Variants for Inhibition of AZTMP Excision In Vitro and for Inhibition of HIV RT-Catalyzed DNA Synthesis In Vitro

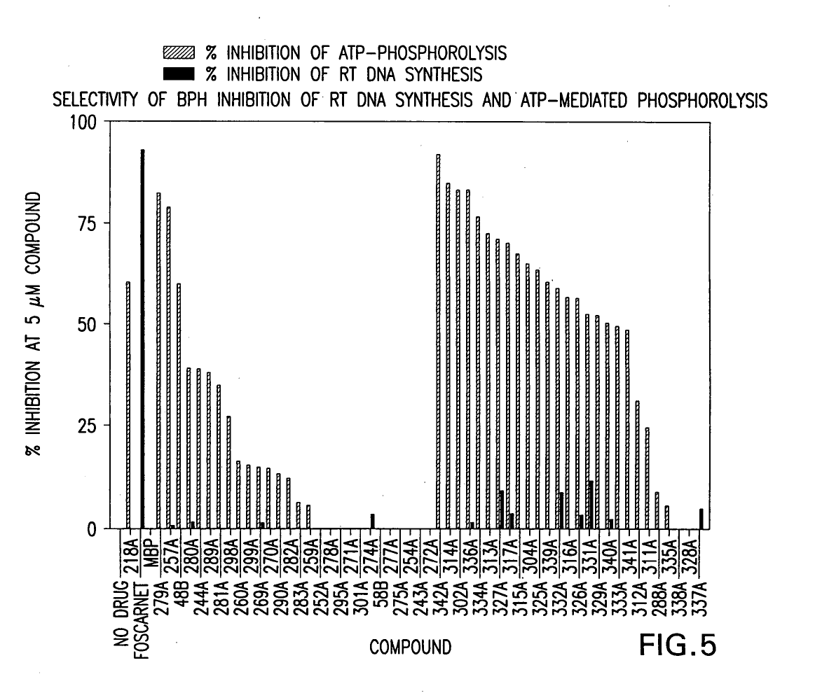

The [³²P]-labeled, 3′-AZTMP-terminated nucleic acid excision substrate (“AZTMP-T/P”) is made as described in Example 2. AZTMP-T/P is mixed with a 3-fold molar excess of TAM-mutant HIV-1 reverse transcriptase in 50 mM Tris, pH 8.1, containing 60 mM KCl and 10 mM MgCl₂ and incubated for 5 minutes at 37° C. to allow formation of the RT-AZTMP-T/P binary complex. Specific concentrations of BPH variants are added (typically sufficient to provide 5 μM (as in FIG. 5) to 50 μM final concentration), then reactions are initiated by the addition of ATP (to a final concentration of 3 mM) or sodium pyrophosphate (to a final concentration of 50 μM). After 10 minutes (for reactions with ATP) or 5 minutes (for reactions with sodium pyrophosphate), the reactions are stopped by the addition of 2 volumes of sequencing gel loading buffer (98% deionized formamide, 10 mM EDTA, and 1 mg/ml each of bromophenol blue and xylene cyanol). An identical control reaction in which RT is omitted is also carried out simultaneously. Samples are heated at 100° C. for 5 minutes then subjected to polyacylamide gel electrophoresis using 16% polyacrylamide/7 M urea gels. Electrophoretically resolved products are visualized and quantified by phosphorimaging analysis. The percent excision is defined by the amount of the 19 nucleotide [³²P]-labeled, 3′-AZTMP-terminated primer DNA in the sample divided by the amount of the 19 nucleotide [³²P]-labeled, 3′-AZTMP-terminated primer DNA in the control, multiplied by 100.

HIV-1 RT DNA polymerase activity (RT-catalyzed DNA synthesis) is determined by a fixed time assay. Reaction mixtures (50 μl total volume) contain 50 mM Tris-HCl (pH 7.8, 37° C.), 60 mM KCl, 10 mM MgCl₂, 5 μg/ml of either poly(rA)-oligo(dT)₁₂₋₁₈ and 20 μM [³H]TTP. Specific concentrations of BPH variants are added (typically sufficient to provide 5 μM (as in FIG. 5) to 50 μM final concentration), then reactions are initiated by the addition of RT. Reaction mixtures are incubated at 37° C. for 20 min and then quenched with 250 μl of ice-cold 10% trichloroacetic acid containing 20 mM sodium pyrophosphate. Quenched samples are left on ice for 20 min, then filtered using 1.2 μm glass fiber Type C filter multi-well plates (Millipore), and washed sequentially with 10% TCA containing 20 mM sodium pyrophosphate and with 100% ethanol. The extent of radionucleotide incorporation is determined by liquid scintillation spectrometry of the dried filters.

FIG. 5 shows the results of screening bisphosphonate variants at 5 μM final concentration for inhibition of AZTMP excision in vitro and for inhibition of HIV RT-catalyzed DNA synthesis in vitro. It is evident that the different bisphosphonate variants have significant but different potency for inhibition of HIV RT-catalyzed AZTMP excision (ATP-mediated bars in FIG. 5). It is also evident that these same bisphosphonate variants possess little or no inhibitory potency against HIV RT-catalyzed DNA synthesis. The bisphosphonate variants are therefore specific inhibitors of HIV RT-catalyzed AZTMP excision.

### Example 6

### Method for Screening of Bisphosphonate Variants for Antiviral Activity Against AZT-Resistant HIV in the Absence and in the Presence of AZT

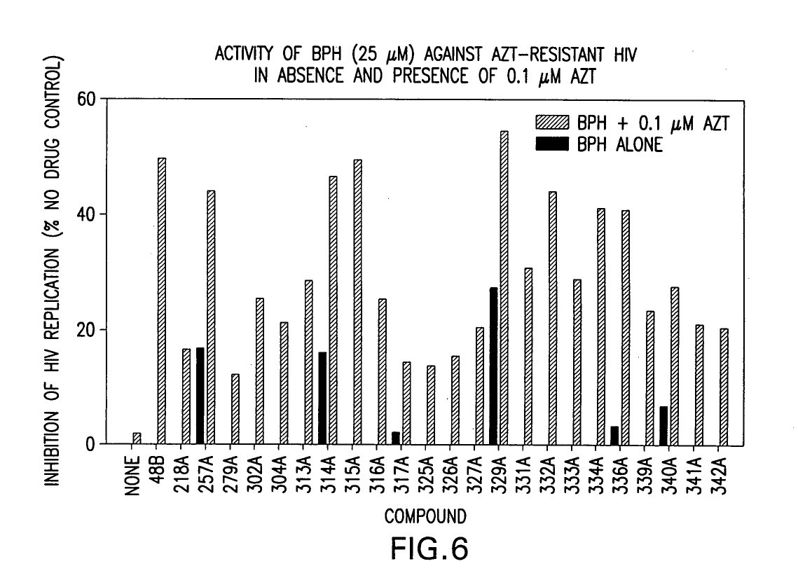

P4/R5 cells are plated in 96-well culture dishes at a cell density of 3×10³ cells/well in 100 μl DMEM/10% fetal bovine serum. In one set of plates, bisphosphonate variants are added (25μM final concentration in FIG. 6) to individual wells. In another identical set of plates, bisphosphonate variants are added along with specific concentrations of AZT (0.1 μM final concentration in FIG. 6). Each well is inoculated with AZT-resistant HIV (multiplicity of infection=1), and the cells are incubated at 37° C. for 48 hours. The cells are then washed and the extent of HIV infection is determined fluorometrically. The percent inhibition of HIV replication is calculated as [1-(fluorescence signal of cells infected in the presence of bisphosphonate divided by the fluorescence signal of cells infected in the absence of any drug)] multiplied by 100.

FIG. 6 shows that AZT (at 0.1 μM final concentration) has essentially no antiviral activity against AZT-resistant HIV (2% inhibition in FIG. 6). This figure also shows that most bisphosphonate variants have little or no antiviral activity in the absence of AZT. Although each of AZT and the BPH are inactive alone, significant antiviral activity is noted when bisphosphonate variants are combined with AZT.

### Example 7

### Toxicity of BPH Compounds

BPH alone or in combination with AZT is orally administered repeatedly to male SD rats (Sprague-Dawley rats) and male beagle dogs for 14 days to evaluate the BPH toxicity.

Administered to SD rats is 0, 50, 500, 1000, and 2000 mg/kg of the BPH alone in single use (5 rats per group). (5 rats per group). In combination, AZT is administered at from 10-2000 mg/kg.

Administered to beagle dogs is 0, 50, 500, 1000, and 2000 mg/kg of the BPH alone in single use (3 dogs per group). In combined use, AZT is administered in the amounts of 10-2000 mg/kg (3 dogs per group).

The present invention is not to be limited in scope by the specific embodiments described above. Many modifications of the present invention, in addition to those specifically recited above would be apparent to the skilled artisan using the teachings of the instant disclosure. Such modifications are intended to fall within the scope of the appended claims. All publications, patents and patent publications, cited above are herein incorporated by reference in their entireties.

1. A composition comprising a bisphosphonate and a nucleoside reverse transcriptase inhibitor.

2. The composition of claim 1 wherein the nucleoside reverse transcriptase inhibitor is 3′-azido-3′-deoxythymidine.

3. The composition of claim 1 wherein the bisphosphonate has the formula (I): 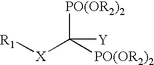
where X=NH, CH₂, CF₂, O, S
Y=H, OH
R₁=an aryl group, an arylalkyl group, an aryloxy group, an acyloxy group, an alkyl group, an alkenyl group, an alkynyl group, an alkoxy group, a haloalkyl group, a perfluoroalkyl group, a carbamoyloxy group, a cyanoalkyl group, an azidoalkyl group, a hydrazinoalkyl group, a hydroxyalkyl group, an alkoxyalkyl group, an aminoalkyl group, an alkylaminoalkyl group, a dialkylaminoalkyl group, an aryl aminoalkyl group, a diarylaminoalkyl group, an arylalkyl aminoalkyl group.
R₂=H, a methylene group, a carbonate group.

4. The composition of claim 1 wherein the bisphosphonate is selected from the group consisting of Compound Nos. 48B, 215A 218A, 228A 244A, 248A, 250A, 251A, 253A, 255A, 256A, 257A, 262A, 279A, 280A, 281A, 289A, 298A, 302A, 304A, 311A, 312A, 313A, 314A, 315A, 316A, 317A, 325A, 326A, 327A, 329A, 331A, 332A, 333A, 334A, 336A, 339A, 340A, 341A, and 342A.

5. A composition comprising a substantially isolated complex of a bisphosphonate and a HIV fragment that binds the bisphosphonate.

6. A method for modulating nucleoside reverse transcriptase inhibitor excision, inhibiting retroviral replication, or restoring the antiviral activity of a nucleoside reverse transcriptase inhibitor in a cell, the method comprising contacting the cell with an effective amount of a bisphosphonate and a nucleoside reverse transcriptase inhibitor, wherein the bisphosphonate inhibits nucleoside reverse transcriptase inhibitor chain termination excision.

7. The method of claim 6 wherein the cell is infected with a nucleoside reverse transcriptase inhibitor-resistant retrovirus.

8. A method for prolonging the use of a nucleoside reverse transcriptase inhibitor for the treatment of a nucleoside reverse transcriptase inhibitor-resistant antiviral disorder in a subject comprising administering to the subject an effective amount of a bisphosphonate, wherein the bisphosphonate inhibits nucleoside reverse transcriptase inhibitor chain termination excision.

9. The method of claim 8 wherein the nucleoside reverse transcriptase inhibitor is 3′-azido-3′-deoxythymidine.

10. The method of claim 8 wherein the antiviral disorder is HIV.

11. A method for sensitizing a subject having a retrovirus-related disorder to treatment with a nucleoside reverse transcriptase inhibitor, the method comprising administering to the subject an effective amount of a bisphosphonate, wherein the bisphosphonate inhibits nucleoside reverse transcriptase inhibitor chain termination excision.

12. A method for inhibiting retroviral replication, reducing viral titer, treating a retrovirus-related disorder, or reducing the occurrence of secondary infections in a subject, the method comprising administering to the subject an effective amount of a bisphosphonate and a nucleoside reverse transcriptase inhibitor, wherein the bisphosphonate inhibits nucleoside reverse transcriptase inhibitor chain termination excision.

13. The method of claim 12 wherein the effective amount of a nucleoside reverse transcriptase inhibitor is a reduced dose when compared to standard dosages for nucleoside reverse transcriptase inhibitor treatment.

14. The method of claim 12 wherein the administering of the nucleoside reverse transcriptase inhibitor is for a short period of time or for few treatment cycles when compared to standard treatment times and cycles for nucleoside reverse transcriptase inhibitor treatment.

15. The method of claim 12 further comprising administering another antiviral compound, wherein the administering of the other antiviral compound is for a short period of time or for few treatment cycles when compared to standard treatment times and cycles for treatment using the other antiviral compound.

16. A method for identifying an agent that modulates nucleoside reverse transcriptase inhibitor excision by a bisphosphonate-based excision inhibitor, the method comprising: (1) contacting a test compound with a composition comprising a substantially isolated complex of a bisphosphonate and a bisphosphonate-recognition site; and (2) determining the presence or absence of a modulating effect on bisphosphonate-mediated nucleoside reverse transcriptase inhibitor excision, wherein demonstration of the modulating effect indicates that the test compound is an agent that modulates nucleoside reverse transcriptase inhibitor excision by the bisphosphonate.
